(19) United States
(12) Patent Application Publication
(10) Pub. No.: US 2021/0393850 A1
(43) Pub. Date: Dec. 23, 2021

(54) BIOINKS

(71) Applicants: UNIVERSITEIT MAASTRICHT, Maastricht (NL); ACADEMISCH ZIEKENHUIS MAASTRICHT, Maastricht (NL)

(72) Inventors: Matthew Brandon BAKER, Maastricht (NL); Shahzad HAFEEZ, Maastricht (NL)

(21) Appl. No.: 17/292,028

(22) PCT Filed: Nov. 7, 2019
(86) PCT No.: PCT/EP2019/080507

§ 371 Date: May 7, 2021

(30) Foreign Application Priority Data
Nov. 7, 2018 (EP) 18205041.9

(51) Int. Cl. A61L 27/20; C08L 5/04; A61L 27/38; A61L 27/52; C08J 3/075; C08J 3/24; B33Y 70/00; B33Y 80/00; B29C 64/106

(52) U.S. Cl. CPC A61L 27/20; C08L 5/04; A61L 27/38; A61L 27/52; C08J 3/075; B33Y 10/00; B33Y 70/00; B33Y 80/00; B29C 64/106; C08J 2305/04; B29K 2005/00; C08J 3/24

(57) ABSTRACT

The present invention relates to a hydrogel comprising an oxidized alginate containing aldehyde groups, wherein the oxidized alginate is crosslinked with an imine type crosslinker. The hydrogel is particularly suitable as a bioink, i.e. for 3D printing of cell structures. The gels provide good printability and exhibit excellent viscoelasticity, shear thinning and self-healing characteristics.

The present invention relates to a hydrogel comprising an oxidized alginate containing aldehyde groups. The hydrogel is particularly suitable for printing 3D structures, in particular for 3D printing structures that contain cells.

Tissue engineering is using cells, supporting material—scaffolds, growth factors and in many cases bioreactors, to grow in vitro or in vivo tissue and organs. Cells should have a three-dimensional (3D) environment similar to a native tissue environment to be able to migrate, proliferate and/or differentiate to develop functional tissues. This is the reason why scaffolds with 3D architecture and specific microporosity have been developed for tissue engineering applications. In classical tissue engineering experiments, cells are seeded in a 3D scaffold and then cultivated in an incubator or stimulated in a bioreactor or directly implanted in vivo.

T. Andersen et al, describe in Microarrays, 2015, 4, 133-161 that there is a need for growing cells not only in a 2D environment but providing a 3D cell culture. Nearly all cells that make up tissue reside in an extracellular matrix (ECM). The ECM consists of a complex three dimensional (3D) fibrous meshwork of collagen and elastic fibers embedded in a highly hydrated gel-like material of glycoasminoglycans, proteoglycans and glycoproteins. There is a need for biomaterials that can mimick several of the characteristics of the ECM.

Many different synthetic and natural polymers have been evaluated as a scaffold for tissue engineering. While these materials can be fabricated into films, meshes or more complex 3D structures, their successful use is limited by their physical and biochemical properties.

An alternative for creating 3D structures is 3D printing. In 3D printing processes an object is fabricated layer by layer by a printer device using computer aided design (CAD). Scaffolds made of thermoplastic polymers have been extruded using 3D printers. The disadvantage of 3D printing is the difficulty in cell seeding due to a limited cell migration into porous structures.

3D bioprinting techniques such as ink-jet and extrusion thus have the need for biocompatible “inks” that have the ability to print customizable self-supporting cell-laden structures for soft tissues. To mimic the extracellular matrix, viscoelastic properties are needed. The article of T. Andersen in Microarrays mentioned above provides an overview of technologies available at this time (page 148-149).

US2016/0279868 describes a method of manufacturing a 3D structure by delivering first ink into a template material wherein the template material comprising a self-healing supramolecular gel that can maintain the shape and dimensional stability of the first ink. The supramolecular gel comprises hyaluronic acid preferably functionalized with β-cyclodextrin.

WO00/21572 describes hydrogels and water soluble carriers for drug delivery. The hydrogels are based on alginate that is oxidized to convert at least a portion of the guluronate units to aldehyde guluronated units. In order to be suitable for this application, the molecular weight of the alginate is reduced such that the materials can be eliminated from the body after degradation of the crosslinking.

The goal of the present invention is to provide dynamic covalent gels for printability, which exhibit excellent viscoelasticity, shear thinning and self-healing characteristics.

The present invention thus provides a hydrogel comprising an oxidized alginate containing aldehyde groups, wherein the oxidized alginate has a weight average molecular weight Mw of 80,000 daltons or more and the oxidized alginate is crosslinked with an imine type crosslinker selected from:

an alkoxy compound having formula (I):

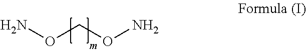

wherein m is 2 to 12,

a semicarbazide compound having formula (II):

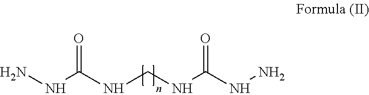

wherein n is 2 to 12, and

a hydrazide compound having formula (III):

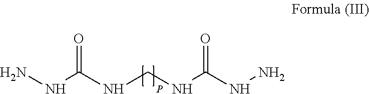

wherein p is 2 to 12

wherein

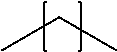

represents an alkylene group having m, n or p carbon atoms, wherein 1 or more carbon atoms can be replaced by a heteroatom selected from 0, S and N;
a compound having formula (IV):

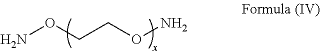

wherein x is 1 to 20;

a compound having formula (V):

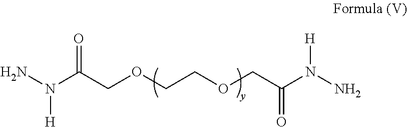

wherein y is 1 to 20;

a compound having formula (VI):

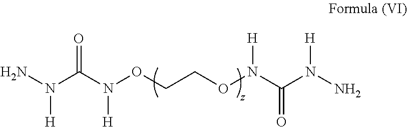

wherein z is 1 to 20;

a compound having formula (VII)

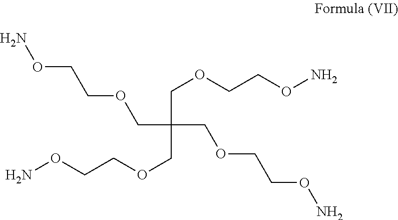 [SMILES: NOCCOCC(COCCON)(COCCON)COCCON]

a compound having formula (VIII)

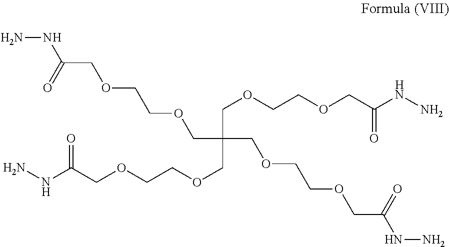 [SMILES: NNC(=O)COCCOCC(COCCOCC(=O)NN)(COCCOCC(=O)NN)COCCOCC(=O)NN]

a compound having formula (IX):

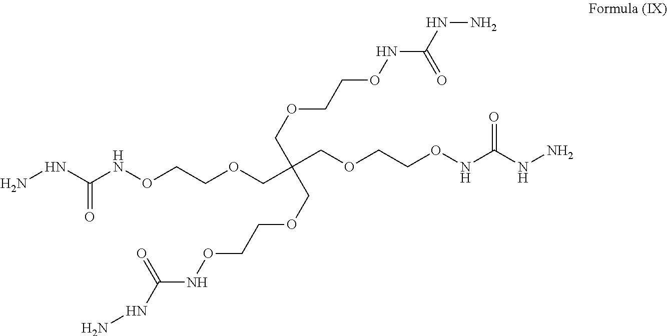 [SMILES: NNC(=O)NOCCOCC(COCCONC(=O)NN)(COCCONC(=O)NN)COCCONC(=O)NN]

a compound having formula (X):

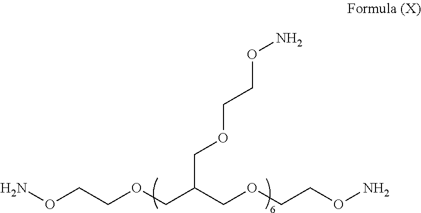

a compound having formula (XI)

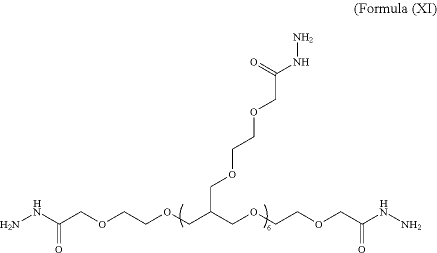

and

a compound having formula (XII):

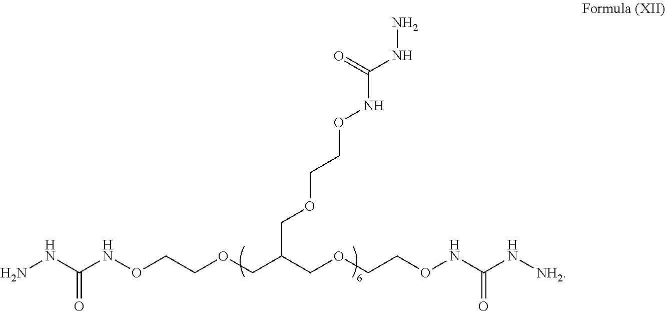

In the above compounds of formula's I to III

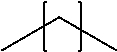

preferably represents an alkylene group having m, n or p carbon atoms.

The alkylene group can be straight or branched. Preferably the alkylene group is straight.

In Formula I, m is preferably 2 to 5, more preferably 3 or 4, most preferably 3. In Formula II, n is preferably 2 to 10, more preferably 4 or 8, most preferably 6. In Formula III, p is preferably 3 to 5, more preferably 4.

The NH₂ end groups in the above compounds of formula's I to XII can optionally be substituted by a functional group. Preferably, the NH₂ end group is not substituted.

Preferably, the oxidized alginate is crosslinked with an imine type crosslinker selected from the group consisting of the compound of Formula (I), the compound of Formula (II) and the compound of Formula (III). Combinations of these crosslinkers are also included.

According to a preferred aspect, the present invention provides a hydrogel comprising an oxidized alginate containing aldehyde groups, wherein the oxidized alginate is crosslinked with an imine type crosslinker selected from an alkoxy compound having structure:

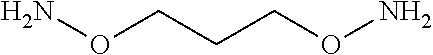

### O,O′-1,3-propanediylbishydroxylamine dihydrochloride

a semicarbazide compound having structure:

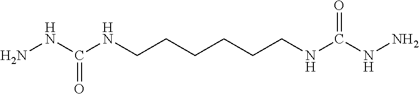 [SMILES: NNC(=O)NCCCCCCNC(=O)NN]

(hexamethylenedisemicarbazide)
or
a hydrazide compound having structure:

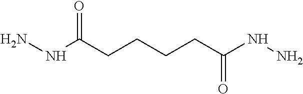 [SMILES: NNC(=O)CCCCC(=O)NN]

(adipic acid dihydrazide)

The advantage of the invention is that it allows to develop a small library of viscoelastic, self-healing and printable hydrogels. By using distinct dynamic covalent chemistries, this platform allows mechanical tenability to better mimic the dynamics of soft tissues while at the same time providing good cytocompatibility and thus cell viability.

In the present application the following terms and definitions are used.

With hydrogel is meant a three dimensional hydrophilic network comprising hydrophilic polymers, in which water is the dispersion medium and that are capable of maintaining their structural integrity.

With oxidized alginate containing aldehyde groups, is meant an alginate wherein the sugar residues have undergone an oxidation reaction creating aldehyde groups:

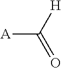

where A is the bond to the alginate.

The oxidation of the alginate material is generally conducted with a periodate oxidation agent, particularly sodium periodate, to provide the alginate with aldehyde groups. The degree of oxidation is controllable by the mole equivalent of oxidation agent, e.g. periodate, to sugar unit. The oxidation level is thus defined as the molar amount of sodium periodate per molar amount of sugar residues in alginate.

The alginate is preferably 2 to 20% oxidized, more preferably 5 to 15%.

This oxidation reaction is shown in FIG. 1(a).

The percentage crosslinking is defined as the molar amount of the respective cross-linker (imine type crosslinker) per molar amount of oxidized alginate repeat units. The extent of crosslinking can be controlled by the concentration of crosslinking agent, the concentration of the oxidized alginate in the solution and the degree of oxidation of the alginate.

The oxidized alginate is preferably 2 to 20% crosslinked, more preferably 5 to 15%.

The alginates used are well-known and are widely commercially available. Alginates are polysaccharides which consist of linear (unbranched) 1,4-linked residues of β-D-mannuronic (M unit) and α-L-guluronic acid (G unit). The alginate molecular structure contains blocks of consecutive G or M units, or blocks of alternating units.

The alginate to be used in the invention preferably has a weight average molecular weight Mw (before oxidation) of between 100,000 and 700,000, preferably between 200,000 and 600,000.

The oxidized alginate preferably has a weight average molecular weight Mw of at least 50,000. The Mw is preferably at least 80,000, more preferably at least 100,000 daltons. Molecular weight can be determined by standard techniques, e.g. by gel permeation chromatography (GPC) analysis with a Shodex PW_{XL} column based on PEG standards using a 0.1 M NaNO₃ solution in water as the eluent.

The hydrogel of the invention can be prepared by the following process:

a) providing an alginate in water to obtain an alginate suspension;
b) adding a periodate to the alginate suspension to oxidize the alginate to create aldehyde groups;
c) adding an imine type crosslinker having a structure of any one of Formulas (I) to (XII) as described above; and
d) allowing the hydrogel to form.
During step a), b) and c) stirring can be applied to ensure correct distribution of the constituents. The alginate suspension in step a) can optionally contain further ingredients, e.g. a buffer. The periodate in step b) is preferably sodium periodate. The periodate is added in an amount of 2 to 20 mol % compared to the molar amount of alginate.

After step b) the oxidized alginate can be purified.

The imine type crosslinker is preferably added in an 0.5-5 times molar amount compared to the molar amount of oxidized alginate obtained in step b. Preferably the imine crosslinker is added in an equimolar amount to the molar amount of oxidized alginate.

It is possible to foresee that other ingredients are added to the hydrogel to optimize its properties. E.g. a catalyst can be added, or an ionic crosslinker such as calcium ions can be added. Further hydrophilic polymers can be mixed with the hydrogel to modify the rheological properties. As shown in the examples, also bioactive ligands, such as an aminooxy terminated RGD peptide, can be added.

In view of the viscoelastic properties of the hydrogel of the invention it is particularly suitable for three dimensional (3D) printing techniques.

In order to use the hydrogel in a printer, it can be included in a reservoir suitable for 3D printing equipment. Such a reservoir has a volume of at least 1 mL. The reservoir can have a volume of at least 2 mL up to 100 mL, preferably up to 50 mL, more preferably up to 10 mL.

According to a further aspect of the invention, a method for manufacturing a 3D hydrogel structure is provided, wherein the method comprises the steps of

a) providing a suspension of an oxidized alginate containing aldehyde groups in a medium;
b) adding an imine type crosslinker selected from the group consisting of

an alkoxy compound having formula (I):

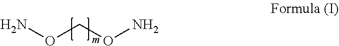

wherein m is 2 to 12,

a semicarbazide compound having formula (II):

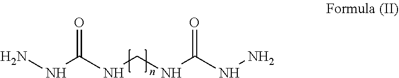

wherein n is 2 to 12, and

a hydrazide compound having formula (III):

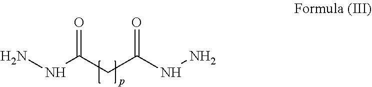

wherein p is 2 to 12

a compound having formula (IV):

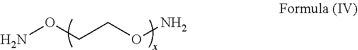

wherein x is 1 to 20;

a compound having formula (V):

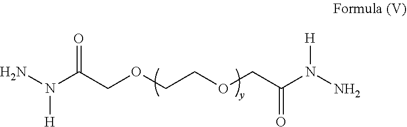

wherein y is 1 to 20;

a compound having formula (VII):

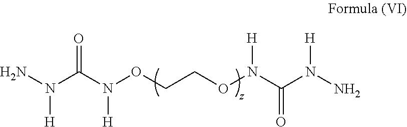

wherein z is 1 to 20;

a compound having formula (VII)

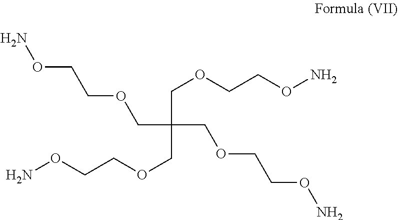 [SMILES: NOCCOCC(COCCON)(COCCON)COCCON]

a compound having formula (VIII)

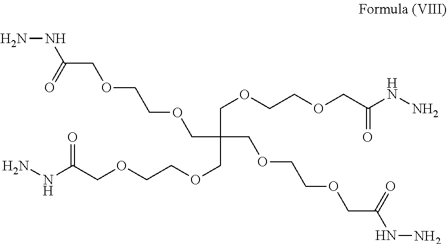 [SMILES: NNC(=O)COCCOCC(COCCOCC(=O)NN)(COCCOCC(=O)NN)COCCOCC(=O)NN]

a compound having formula (IX):

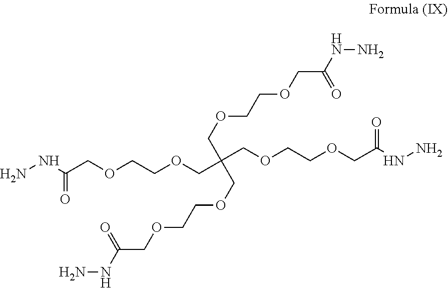 [SMILES: NNC(=O)COCCOCC(COCCOCC(=O)NN)(COCCOCC(=O)NN)COCCOCC(=O)NN]

a compound having formula (X):

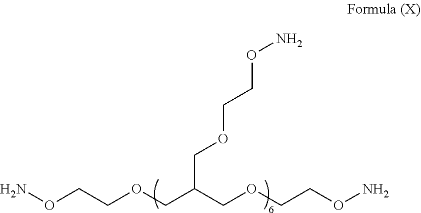

a compound having formula (XI)

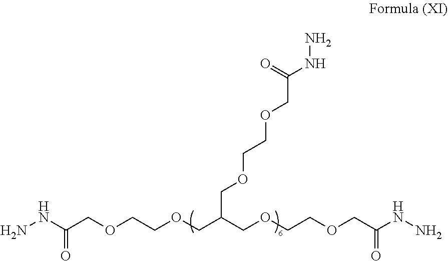

and

a compound having formula (XII):

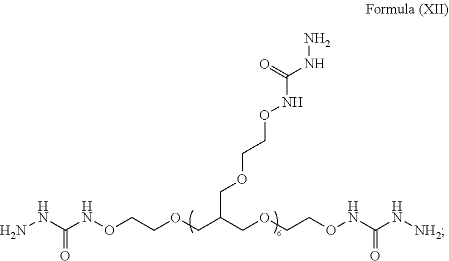

c) allowing a gel to form;
d) depositing the gel obtained in step c) layer by layer on a surface according to a predefined structure.

The medium in step a) is water optionally containing a buffer such as phosphate buffered saline (PBS).

The imine type crosslinker in the method of manufacturing a hydrogel, is preferably selected from a compound having the structure of Formula (I), Formula (II) or Formula (III), more preferably from a compound having the structure:

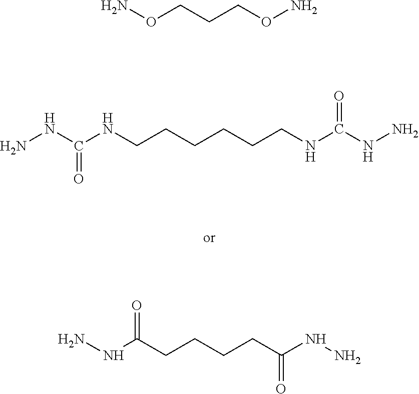 [SMILES: NOCCCON ; NNC(=O)NCCCCCCNC(=O)NN ; NNC(=O)CCCCC(=O)NN]

When manufacturing a 3D hydrogel structure also cells can be added. The cells can be added to the hydrogel after the structure has been created. According to an alternative embodiment, the cells are mixed in step a) of the method with the oxidized alginate. To this end, the oxidized alginate can be in a buffered saline solution, such as Dulbecco's phosphate buffered saline and the cells can be in suspension in a suitable cell medium such as Dulbecco's Modified Eagle Medium (DMEM) with fetal bovine serum (FBS).

When cells are added, step c) of the method above is carried out at about 37° C. in an incubator.

The method above allows to influence the final properties of the hydrogel by influencing several parameters, e.g. the amount of oxidation (between 3 and 20%), the amount of cross-linker (0.5 to 5 times the molar amount of oxidized alginate) and the concentration of the alginate in the medium (between 0.5 and 5% w/v).

The present invention thus also provides a hydrogel as described above, where the hydrogel contains cells.

For depositing the gel layer by layer on a surface, any commercial 3D printer suitable for biomaterials can be used for instance a BioScaffolder (GeSiM-Gesellschaft für Silizium-Mikrosysteme mbH, Germany).

The cells that can be used in the method of the invention are human cells, such as human dermal fibroblasts or animal cells, for instance chondrocytes such as chondrogenic cell line ATDC5. Other cells that can be used are endothelial cells, islet cells and stem cells. The present hydrogels can also be used in encapsulating organoids.

### DESCRIPTION OF THE FIGURES

FIG. 1 shows the ¹H NMR spectrum of alginate with varying degrees of oxidation.

FIG. 2 shows viscoelasticity and self-healing exhibited by a series of 2% (w/v) 10% ox-alg samples prepared with different crosslinkers. a) Frequency sweeps using the three different crosslinkers; oxime, semicarbazone and, hydrazone. b) Strain sweeps from 0% to 800% strain for the same three hydrogels, measured at 10 rad/s.

FIG. 3 shows the effect of degree of oxidation on gel storage and loss modulus and self-healing behavior.

FIG. 4 demonstrates the self-healing capacity of 10% ox-alg samples prepared with semicarbazone and hydrazone crosslinkers.

FIG. 5 shows the effect of different mole equivalents hydrazine crosslinker on storage and loss moduli.

FIG. 6 shows cell viability of chondrocytes cultured in oxime, semicarbazone and hydrazone gels. (a) Image showing live cells (in green) after seven days of encapsulation in gels indicating that great majority of cells are live and gels did not cause cytotoxicity, Scale bar: 200 μm and 25 μm for inset images. (b) Cell metabolic activity recorded after 12, 24, 96, and 168 h. The values reported are an average of n=3, ±standard deviation. * and ** indicates p<0.05 and p<0.01 (Student's t-test, independent sample populations). For “cell aggr” condition, cells were cultured in pellets, a standard condition for chondrocyte cell culture.

FIG. 7 shows human dermal fibroblasts spreading morphologies in oxime RGD ligated gels, (a) on top of (2D) and (b) within (3D) oxime, semicarbazone and hydrazone cross-linked gels after 24 h. Green color represents actin staining and blue color represent nucleus staining. scale bar: 25 μm for 2D, 50 μm for 3D images, and 25 μm for 3D insets.

FIG. 8 shows 2-layered scaffolds printed using 10% ox-alg (2% (w/v) alginate) with hydrazone crosslinks printed at different speeds and pressures. A 0.25 mm (25G) conical needle was used.

FIG. 9 shows a 10% ox-alg (1% (w/v) alginate) hydrazone crosslinked scaffolds of (a) 2 layers and (b) 4 layers printed at 45 kPa (30 mm/s). A 0.20 mm (27G) conical needle was used.

FIG. 10 shows a 2-layered scaffolds printed using 5% ox-alg (2% (w/v) alginate) with hydrazone crosslinks at (a) 45 kPa (10 mm/s), (b) 50 kPa (10 mm/s), and (c) 60 kPa (11 mm/s). A 0.20 mm (27G) conical needle was used.

FIG. 11 shows injectability and bioprintability of (a) 10% ox-alg gels with oxime (a-1), semicarbazone (a-2), and hydrazone (a-3) cross-linkers. The gels were extruded through a 25G needle. Semicarbazone and hydrazone showed injectability, and to our surprise oxime gels were also injectable, although they were not self-healing. (b) Printed MERLN and vascular tree structures using hydrazone crosslinks; 2% (w/v) of the 10% ox-alg used for (b-2-b-6) and 3% (w/v) and 4% (w/v) of 5% ox-alg used for (b-7) and (b-8), respectively. From the top left: 3D model of MERLN structure (b-1), printed MERLN structure (b-2, 6 mm thickness), 3D model of vascular tree (b-3), printed vascular tree (b-4, 2 mm thickness), printed vascular tree (b-5, 6 mm thickness), printed vascular tree (b-6-b-8) where the network was manually disrupted (fluorescein included for visibility), scale bar: 5 mm. (c) 10% ox-alg hydrazone gels after 24 h, (c-1): Without printing and (c-2): After bioprinting. Green color represents live cells and red color represents dead cells, Scale bar: 200 μm.

FIGS. 12A and 12B show human mesenchymal stem cells spreading morphology on oxime RGD ligated hydrazone gels. Scale bar: 100 μm for FIGS. 12A and 12B.

FIG. 13 shows the metabolic status of pancreatic islet cells within different hydrogels.

### EXPERIMENTAL

### Materials

Propanoic acid hydrazide (≥90%), O-ethylhydroxylamine hydrochloride (97%), adipic acid dihydrazide (≥98%, AADH), O,O′-1,3-propanediylbishydroxylamine dihydrochloride (98%, PDHA), activated charcoal (Norit), sodium periodate (NaIO4), ethylene glycol, and Dulbecco's modified Eagle's medium-F12 (DMEM-F12, low glucose) were purchased from Sigma-Aldrich. N,N′-(hexane-1,6-diyl)bis(hydrazinecarboxamide) (HDCA) was synthesized according to Sims, M. B.; Patel, K. Y.; Bhatta, M.; Mukherjee, S.; Sumerlin, B. S. Harnessing Imine Diversity to Tune Hyperbranched Polymer Degradation. Macromolecules 2018, 51, 356-363, doi:10.1021/acs.macromo1.7b02323.

Dulbecco's phosphate buffer saline (PBS), fetal bovine serum (FBS), penicillin/streptomycin (P/S), calcein AM, ethidium homodimer and PrestoBlue™ cell viability reagent were purchased from Thermo Fisher Scientific. Dialysis membrane (Spectra/Por®) with molecular weight cut off (MWCO) 3500 Dalton (Da) was obtained from VWR, Netherlands.

Sodium alginate was purchased from FMC (Manugel GMB, Lot No. G9402001). Mn was determined as 258,439 and Mw was determined as 518,701.

RGD used was prepared according to Zamuner, A.; Cavo, M.; Scaglione, S.; Messina, G. M. L.; Russo, T.; Gloria, A.; Marietta, G.; Dettin, M. Design of decorated self-assembling peptide hydrogels as architecture for mesenchymal stem cells. Materials (Basel). 2016, 9, doi:10.3390/ma9090727 and had the following structure:

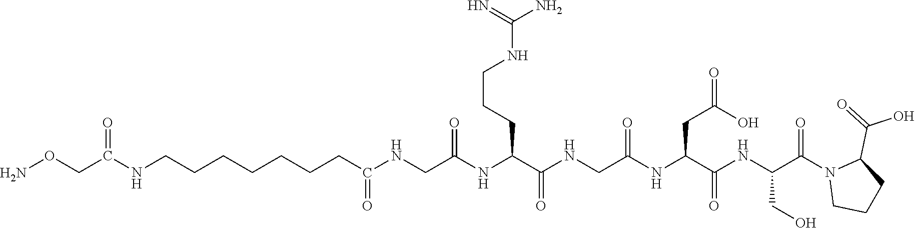 [SMILES: N=C(N)NCCC[C@H](NC(=O)CNC(=O)CCCCCCCNC(=O)CON)C(=O)NCC(=O)N[C@@H](CC(=O)O)C(=O)N[C@@H](CO)C(=O)N1CCC[C@@H]1C(=O)O]

Based on high temperature NMR (370K), estimation for G and M blocks in this alginate is 74% and 26% respectively. Calculations carried out on estimations of block compositions were based on the report by Grasdalen (Grasdalen, H. High-Field, 1H-N.M.R. Spectroscopy of Alginate: Sequential Structure and Linkage Conformations. Carbohydr. Res. 1983, 118, 255-260) and Penman (Penman, A.; Sanderson, G. R. A Method for the Determination of Uronic Acid Sequence in Alginates. Carbohydr. Res. 1972, 25 (2), 273-282.)

### Alginate Purification

Alginate powder was dissolved in deionized (DI) water at a concentration of 1% (w/v). Activated charcoal (1% (w/v)) was added, and the alginate solution was stirred for 24 h at 4° C. Subsequently, the alginate solution was filtered with 11 μm, 1.2 μm, 0.45 μm, and 0.2 μm Whatman membrane filters to remove charcoal particles. The alginate solution was then frozen and lyophilized.

### Synthesis of Oxidized Alginate

### Example 1; 5% Oxidation

Purified alginate (1.00 g, 5.68×10⁻³ mol monomer) was dissolved in 100 mL DI water. Keeping the reaction in the dark, sodium periodate (6.07×10⁻² g, 2.84×10⁻⁴ mole) was added in one portion with stirring. After 17 hours in the dark (at RT), the reaction was quenched by addition of ethylene glycol (1.76×10⁻² g, 2.84×10⁻⁴ mole, equimolar to NaIO₄). The reaction solution was stirred in dark for a 1 hr to stop the reaction completely. The reaction solution was dialyzed against water using membrane tubes of MWCO 3500 Da (3 days with 3× water change per day), then lyophilized yielding a white fibrous material (above 80% yield).

### Example 2, 10% Oxidation

A similar procedure as Example 1 was followed, except that (1.21×10⁻¹ g, 5.68×10⁻⁴ mole) sodium periodate was used. Yield was above 80%:

### Example 3, 15% Oxidation

A similar procedure as Example 1 was followed, except that (1.82×10⁻¹ g, 8.52×10⁻⁴ mole) sodium periodate was used. Yield was above 80%:

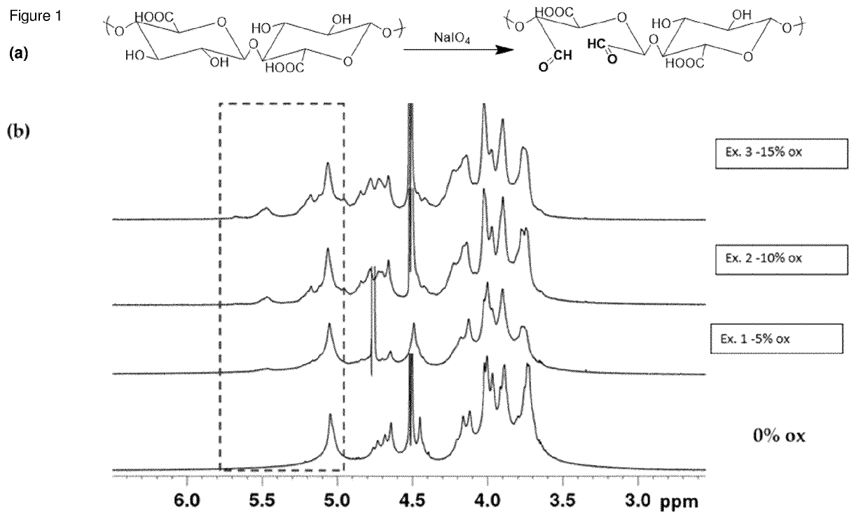

The oxidation of alginate in Examples 1 to 3 was confirmed by the presence of hemiacetal peaks in the NMR spectra and it was found that with an increase in the degree of oxidation, the hemiacetal peak intensity increased. This is shown in FIG. 1: The appearance of protons between 5.15-5.75 ppm (highlighted in the box) is attributed to the formation of hemiacetal groups upon reaction of aldehydes to neighboring hydroxyl groups. Spectra were measured at 325 K.

The molecular weight of the oxidized alginates of Examples 1, 2 and 3 was tested with a Shodex PW_{XL} 4000 column (MW up to about 300,000 based on PEG standards), using a 0.1 M NaNO₃ solution in water as the eluent. Samples were prepared in water with 0.1 M NaNO₃ at concentrations of about 5 mg/ml.

For Example 1 (5% ox), Mₙ was 89,000, M_{w} was 247,100 and polydispersity was 2.77.

For Example 2 (10% ox), Mₙ was 69,900, M_{w} was 205,300 and polydispersity was 2.94.

For Example 3 (15% ox), Mn was 56,600, M_{w} was 167,000 and polydispersity was 2.95.

### Preparation of Hydrogels

Oxidized alginate samples were weighed into 1.5 mL Eppendorf tubes. To prepare alginate solutions of 2.5% (w/v), PBS was added and the solution was mixed for 30 min on a thermoshaker at RT (2000 rpm). Unless indicated otherwise, crosslinker solution was added (prepared in PBS) to prepare hydrogels with equimolar concentrations of aldehyde/crosslinker functionalities with a final alginate concentration of 2% (w/v).

Solutions were quickly transferred to polydimethylsiloxane molds with a disc geometry of 12 mm in diameter and 2.0 mm in thickness. Coverslips were placed on top of gel solutions during the gelation process to ensure hydrogels have flat top surfaces and homogeneous thickness. Gelation was left to occur for 30 min at room temperature and then overnight at 4° C. before rheological measurements were carried out.

| Example reference | Oxidized alginate | Crosslinker |
|---|---|---|
| 5% ox - Hydrazone | Example 1 | AADH |
| 5% ox - Oxime | Example 1 | PDHA |
| 5% ox - Semicarbazone | Example 1 | HDCA |
| 10% ox - Hydrazone | Example 2 | AADH |
| 10% ox - Oxime | Example 2 | PDHA |
| 10% ox - Semicarbazone | Example 2 | HDCA |
| 15% ox - Hydrazone | Example 3 | AADH |
| 15% ox - Oxime | Example 3 | PDHA |
| 15% ox - Semicarbazone | Example 3 | HDCA |

All experiments resulted in self-standing hydrogels.

### Measurements

### Rheological Measurements

Rheological measurements of hydrogels were carried out using an Anton Paar MRC 702 at 23° C. using a parallel plate geometry with bottom and top diameters of 50 mm and 12 mm respectively. During loading, the experimental gap size was set when a threshold normal force was reached; 1 N for stiffer gels and 0.1 N for softer gels. This ensures good contact with the plates, prevents slippage, and increases the sensitivity of measurements by increasing the torque response. Samples were protected against evaporation by the addition of 2-3 drops of distilled water.

Oscillatory strain amplitude sweeps were conducted with strains from 1% to 800% at a frequency of 10 rad/s. Oscillatory frequency sweeps were performed from either 0.1 rad/s or 1 rad/s up to 100 rad/s. Step-strain measurements were undertaken to evaluate the self-healing capacity of hydrogels. Samples were subjected to 3 cycles, each consisting of 1% strain at 10 rad/s for 180 seconds followed by 600% strain at 10 rad/s for 100 seconds.

### Cell Encapsulation/Seeding and Viability Assays

ATDC5 chondrocytes cells were cultured at 37° C. under a 5% CO₂ atmosphere in Dulbecco's Modified Eagle's Medium-F12 (DMEM-F12, low glucose) supplemented with 10% (v/v) fetal bovine serum (FBS) and 1% (v/v) pen/strep. Cells were washed with PBS, trypsinized (0.05%), centrifuged, and then resuspended in a 10% ox-alg solution. They were subsequently mixed with crosslinker to yield a homogenous mixture of 2% (w/v) alginate containing 4×10⁶ cells/mL. 50 μl of the alginate mixture was transferred to a polydimethyl siloxane (PDMS) mold with a disc geometry of 12 mm in diameter and 2.0 mm in thickness. Gels were transferred to a non-adherent 24-well plate. Dulbecco's Modified Eagle's Medium-F12 (DMEM-F12, low glucose) supplemented with 10% (v/v) fetal bovine serum (FBS) and 1% (v/v) pen/strep was refreshed every two days.

### Viability Assays

For live-dead assays, cells were stained with calcein AM/ethidium homodimer Live/Dead solution according to the manufacturer's instructions on days 1, 4 and 7. For the metabolic assay, PrestoBlue solution (10% (v/v) in DMEM-F12, supplemented with FBS) was added and cells were incubated for 2 h in the dark at 37° C. At each time point, 100 μl was collected from each well and the fluorescence intensity was recorded using microplate reader at an excitation wavelength of 530 nm and an emission wavelength of 590 nm.

### Cell Spreading

Alginate gels were prepared as described in section above. Briefly, oxidized alginate was dissolved in PBS and aminooxy terminate RGD peptide was coupled to the network using oxime ligation chemistry. The final RGD ligand concentration was set to 1000 μM with 2% (w/v) oxidized alginate. Equimolar concentrations of crosslinkers (relative to aldehyde functionalities) were added and mixed uniformly. 25 μl of the oxidized alginate mixture was transferred to a 96 flat bottom black well plate, centrifuged at 1500 RCF for 5 minutes to form a uniform layer on the bottom and left to crosslink for 45 minutes. HDF cells were seeded on top of the gel with a cell density of 100 k/gel and cells were fixed using 4% PFA prior to imaging.

### Bioprintability Viability

For cell viability (live-dead) after bioprinting, 10% ox-alg hydrazone hydrogels containing an ATDC5 cell suspension were prepared as mentioned above. A cell-laden solution was loaded into a 3 mL cartridge and placed at 37° C. for 1 hr. The cartridge with a G25 conical needle attached to it was then placed onto a BioScaffolder (GeSiM-Gesellschaft für Silizium-Mikrosysteme mbH, Germany). Bioprinting was carried out to create a four corner polygon with a radius of 2.0 mm, comprising 2 meandered strands placed at 1.0 mm apart. Single layer constructs with a height of 0.2 mm were created. The pressure was set to 140 kPa and extrusion was performed at 5.0 mm/s. Scaffolds were printed in 12-well non-treated cell culture plates. Live-dead viability assay were carried out after 24 hours as mentioned above.

### Stiffness

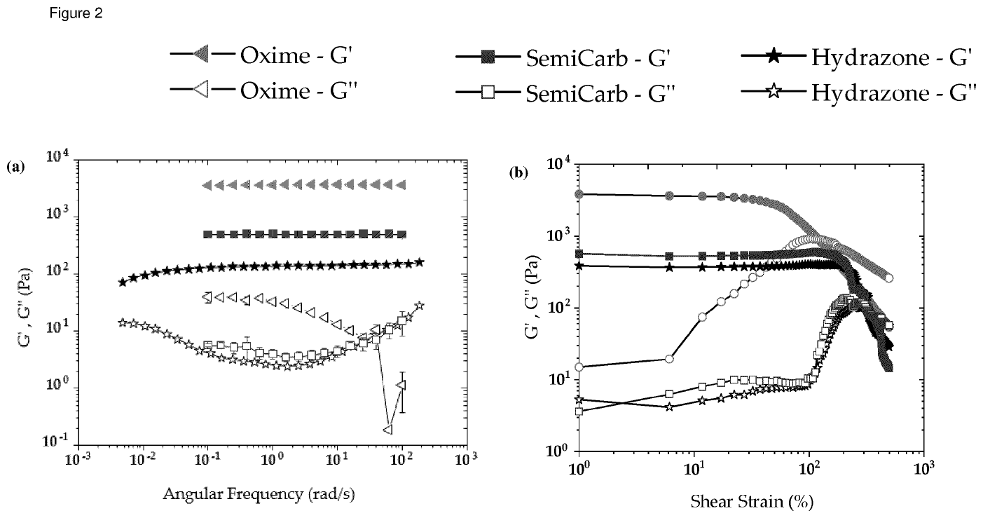

As seen in the frequency sweep plots in FIG. 2a, the oxime gel was the stiffest (G′≈4000 Pa), followed by semicarbazone (G′≈500 Pa), and then hydrazone (G′≈200 Pa). The oxime gel was roughly 8 and 20 times stiffer than semicarbazone and hydrazone, respectively, for an identical formulation (shown in FIG. 2a).

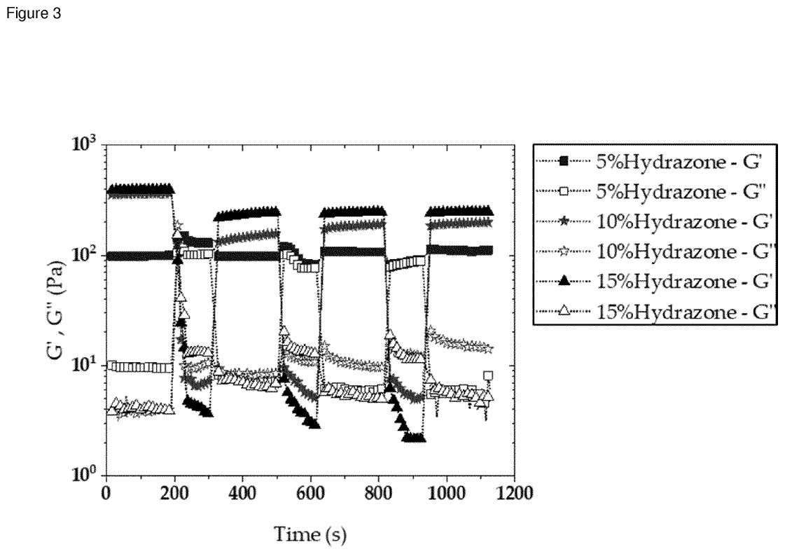

To investigate the effect of the degree of oxidation (DOX) on stiffness, 5% ox-alg, 10% ox-alg and 15% ox-alg gels were made using the hydrazone crosslinker (shown in FIG. 3). Generally, an increase in storage moduli was observed with increasing DOX. Storage moduli increased by a factor of 4 (from ˜90 to ˜350 Pa) as the DOX increased from 5 to 10; however, no considerable difference was observed between 10 and 15% (˜400 Pa).

### Viscoelasticity

The frequency sweep shown in FIG. 2a for both the semicarbazone and the hydrazone gels showed characteristic viscoelastic behavior, with frequency dependent loss moduli over the range explored. While the oxime gel did show some frequency dependence, this appears to represent more elastic behavior. Viscoelastic materials can also show a crossover point between storage (G′) and loss modulus (G″), while moving from rubbery plateau (higher frequency) to terminal region (low frequency). However, this crossover point was not observed even during a longer range scan of the hydrazone gel down to 0.005 rad/s. Typically, low frequencies are needed to observe a crossover point for hydrazone crosslinks However, as such, a crossover point at such small frequencies suggests that the alginate imine type crosslinked hydrogels are very slow stress relaxing networks.

### Self-Healing

Self-healing was initially visualized in the lab for the 15% ox-alg gel. In a glass vial, the gel network was broken using a spatula and self-healing was observed over time via the vial inversion method. The semicarbazone self-healed in≈5-15 minutes while hydrazone took≈30-45 minutes to self-heal. The oxime gel did not self-heal, though interestingly it formed a gel slurry after 72 h, once the network was broken.

To visualize self-healing macroscopically for the 10% ox-alg gel, two disk shape solid gels with different colours (red and green) were made. For a given crosslinker, each pair of gels was cut into 2 halves and then put back into contact within 5 minutes. We observed that the semicarbazone gels self-healed faster than the hydrazone gels and that the oxime gels did not self-heal. After 4 hrs, semicarbazone and hydrazone gels self-healed and the gel boundaries became obscured. Self-healed gels could be stretched using tweezers after 24 hrs: The semicarbazone gel interface stayed stable under stretching force; however, upon overstretching the hydrazone gel showed cracks across the interface.

In order to investigate the self-healing behavior more thoroughly, shear rupture-self-healing cycles were carried out on the rheometer. Initially, stain sweep experiments were carried out to determine the amount of strain needed for gel rupture. These gels proved to be surprisingly tough, with the crossover point for all gels being between 100% and 400% strain (FIG. 2b). Thus, 600% strain was chosen to rupture the 10% ox-alg semicarbazone and hydrazone gel crosslinks and self-healing recovery was allowed under 1% strain.

Upon rupture, the storage and loss moduli inverted and the storage moduli dramatically decreased (<10 Pa). Upon recovery, two-phase self-healing recovery was observed: i) rapid bond reformation under 20 secs upon removing the rupture strain, and ii) a slower recovery of stiffness observed during the next 160 secs. The rapid bond reformation regained a majority of the network stiffness≈70% (820 Pa from 1160 Pa) and≈40% (131 Pa from 356 Pa) of their initial storage moduli compared to≈10% and ≈5% in the slower recovery phase for semicarbazone and hydrazone crosslinks, respectively. Interestingly, crosslinks recover≈10% stronger after 2^{nd} and 3^{rd} rupture cycle compared to 1^{st} rupture cycle.

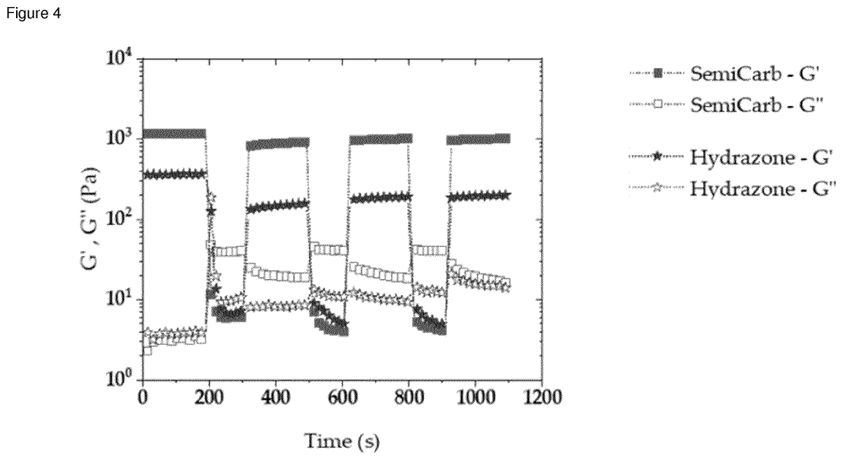

FIG. 4 demonstrates the self-healing capacity of 10% ox-alg samples prepared with semicarbazone and hydrazone crosslinkers. The hydrogels were submitted to three strain cycles. Initially a low strain (1%) was applied, followed by 3 cycles of high strain (600%), to rupture the network, and low strain (1%), to allow recovery. The semicarbazone and hydrazone gels rapidly self-heal.

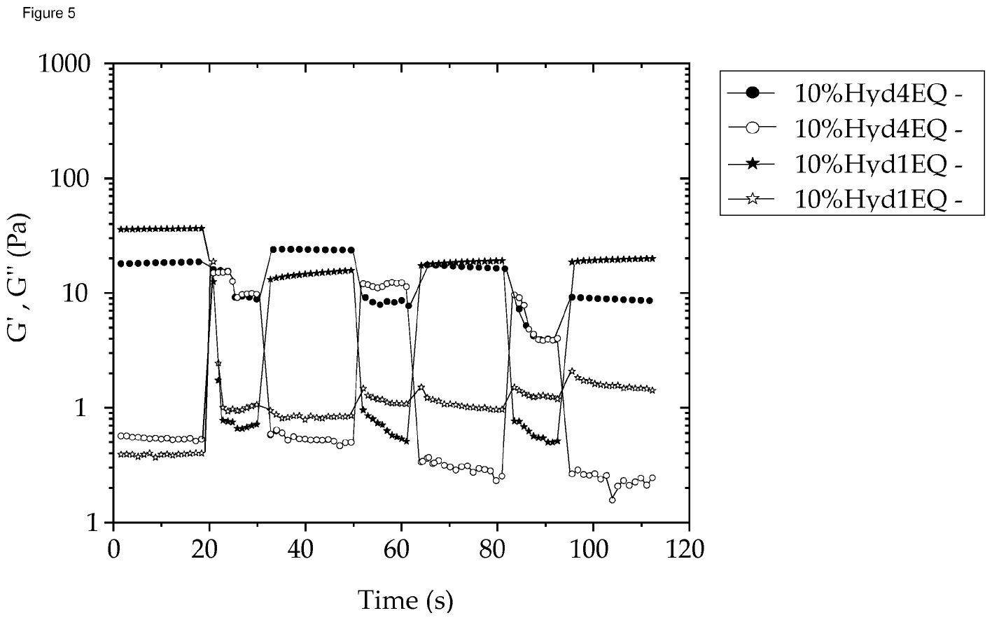

To see the effect of crosslinking density on self-healing, 10% ox-alg with 4-mole equivalents were compared to 1 mole equivalents of hydrazone crosslinks. Immediately apparent, the 1 mole equivalent of crosslinker forms a stiffer gel (350 Pa) with a significantly lower 200 Pa compared to the 4-mole equivalent crosslinker gel. The results are shown in FIG. 5.

### Cell Viability

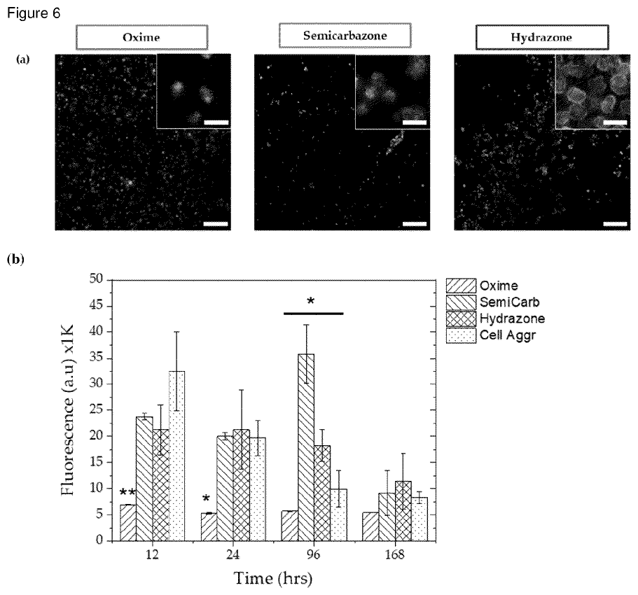

ATDC5 chondrocytes cells were used for cell viability studies (live-dead and metabolic activity) as they are known to survive in gels without biochemical cues (e.g. RGD). ATDC5 were encapsulated (3D) within 10% ox-alg gels and cells were stained and imaged using an inverted fluorescence microscope to evaluate cytotoxicity after 1, 4, and 7 days. Shown in FIG. 6a, great majority of cells were found to be alive in all gels after 7 days. Interestingly, over time, the hydrazone gels appeared to facilitate cell clustering (high magnification images are inset in FIG. 6a). In the semicarbazone and the oxime gels this clustering of cells was noticeably less, which is hypothesized to be a result of less dynamic reorganization of the network. The high multi-day viability of cells within these gels supports future use for cell culture and bioprinting applications.

To confirm the high viability for chondrocytes seen during the live/dead assay, a prestoblue assay was run to investigate the metabolic activity of the cells within the gels over 168 hrs (7 days) (FIG. 6b). Within a series of 2% (w/v) 10% ox-alg gels, the oxime hydrogels maintained significantly lower metabolic activity compared to semicarbazone and the hydrazone hydrogels, which showed similar metabolic activity to the cell aggregate pellet. In particular, chondrocytes showed significantly different metabolic activity in all conditions on day 4. Comparing all gel samples, chondrocytes in the oxime hydrogels showed a consistently low metabolic activity, while other samples started with a high metabolic activity that approximately halved over 7 days. The chondrocytes studied were able to maintain a higher metabolic flux in the more viscoelastic hydrogels (semicarbazone and hydrazone) as compared to the static (oxime) hydrogels.

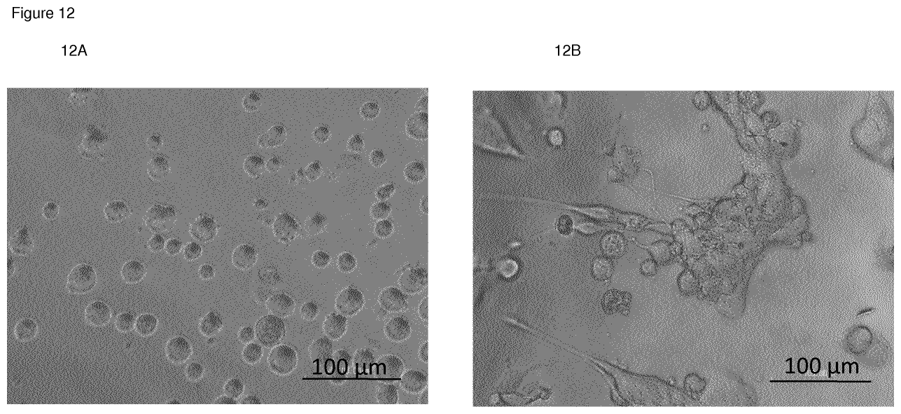

Further, human mesenchymal stem cells (hMSCs) were seeded on top of hydrazone gels. hMSCs had a round morphology after 1 hour (FIG. 12 A) of seeding and showed spreading morphology after 24 hours (FIG. 12B). Spreading morphology is close to their native morphology in human body.

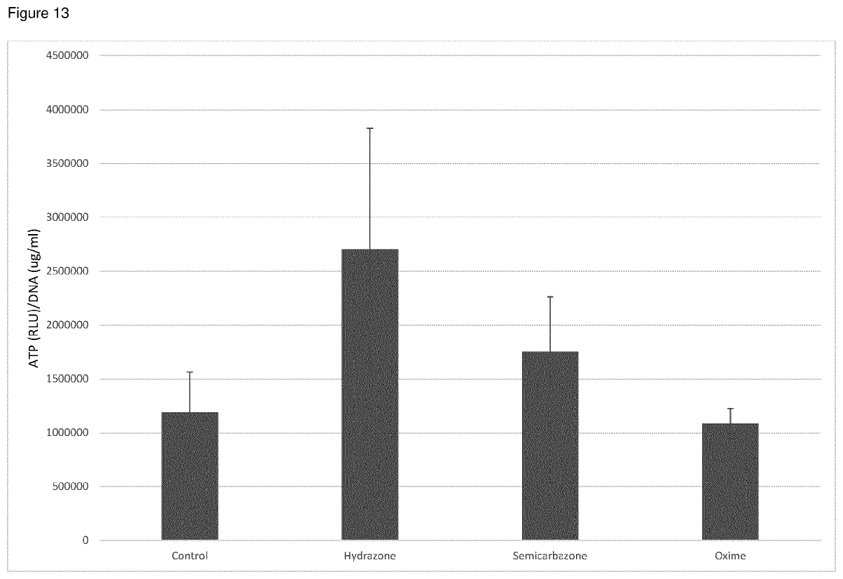

Islet cell aggregates were encapsulated within hydrazone, semicarbazone and oxime hydrogels and results were compared to a positive control (cell aggregates on agarose well plate). Cell metabolic activity was recorded after 24 hours and normalized to total DNA. Islet cell aggregates are more metabolically active in hydrazone gels compared to other gels and a control. The results are shown in FIG. 13.

### Cell Spreading

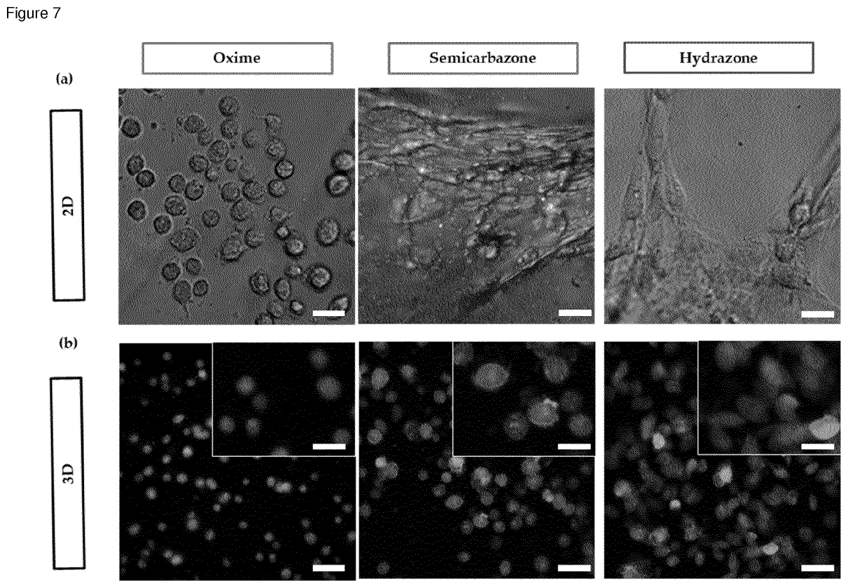

Alginate possesses no active adhesion sites to interact or attach to mammalian cells, but cell adhesion and interaction can be promoted through the conjugation of cell adhesion ligands (e.g. RGD). Hydrogels were biofunctionalized by incorporating aminooxy-terminated RGD peptide (1000 μM) to the 10% ox-alg hydrogel formulation. To investigate whether the crosslinks with different viscoelasticity have an influence on cell spreading, human dermal fibroblasts (HDFs) were seeded on top (2D) for 24 hrs. Cells were imaged in bright field using inverted microscope (see FIGS. 7A and 7B).

We noticed a marked effect on the amount of spreading on the different DCvC crosslinked gels. Cell spreading was found to increase for networks crosslinked by bonds with a higher hydrolysis rate (k₋₁), namely more dynamically rearranging crosslinks. HDFs seeded on the dynamic hydrogels (semicarbazone and hydrazone) showed a spindle shape, increased spreading, and an elongated morphology, with the hydrazone networks showing a more elongated morphology than the semicarbazone. For the oxime hydrogels, only rounded morphologies typical of cells in elastic hydrogels were observed. The same trend was observed with HDF were cultured within gels (3D). Observing larger cell spreading on viscoelastic matrices (semicarbazone and hydrazone) suggests that rearrangement of dynamic crosslinks influenced cell spreading. These results will be further explored in the future and our current hypothesis is that microenvironment clustering of adhesive ligands, or stress relaxation plays a significant role.

Similar results can be shown in a 3D structure.

### Printing

With control over the materials properties, self-healing, cell viability, and biofunctionalization in these materials, we next investigated the injectability of these hydrogels. To assess the shear-thinning capability of the 10% ox-alg with different crosslinkers, we attempted to inject these materials from a syringe through a 25 gauge needle (260 μm i.d.). The hydrazone and semicarbazone hydrogels were injectable through the needle with only the force of the hand and formed intact smooth fibers due to their self-healing capability. Interestingly, the oxime gel was also found to be injectable, but the injected fiber was a non-continuous gel slurry upon injection. Hydrogels made from the 15% ox-alg with both hydrazone and semicarbazone were also observed to be injectable; however, the 15% semicarbazone hydrogels did not produce smooth fibers and required more force for injection. These initial experiments encouraged the exploration for use as bioinks for bioprinting and forecast their suitability as drug or cell delivery vehicles.

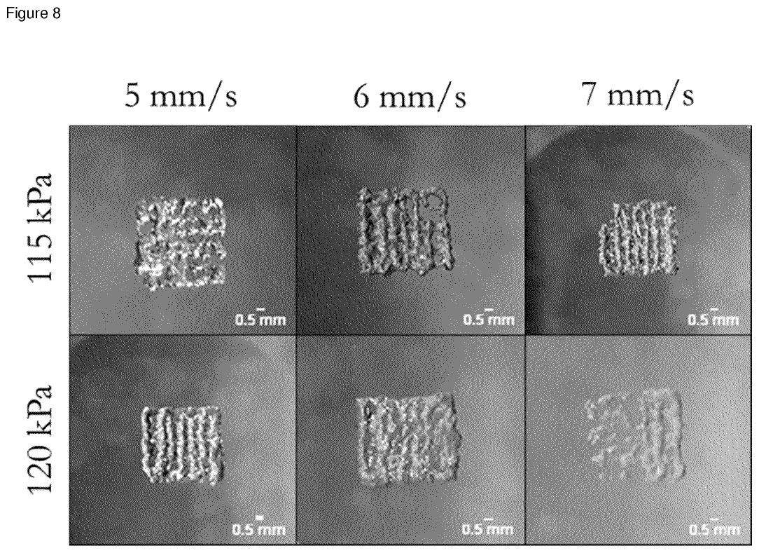

While both hydrazone and semicarbazone gels showed some initial printability, the semicarbazone gels required significantly higher pressures and larger needle diameters. Consequently, the hydrazone gels were deemed more likely to support cell viability and were further optimized for bioprinting. A simple grid structure was employed to study the effects of deposition speed and extrusion pressure on printability. Using different deposition speeds and extrusion pressures, we used the 10% ox-alg hydrazone hydrogels (2% w/v) to deposit 2-layered grid structures via a 0.25 mm diameter conical needle. Extrusion of the hydrogel at 115 and 120 kPa were observed to be more homogeneous and better defined structures were extruded at a speed of 5 mm/s, as opposed to the partial and inhomogeneous hydrogel fibers bioprinted at 7 mm/s (FIG. 8).

This could be expected as the bioprinting speeds employed should allow ample time for appropriate amount of material to be deposited and placed on substrate. Similarly, extrusion pressure was optimized to tune the amount of deposited hydrogel and 3 different pressures (115, 120, and 140 kPa) were tested. A pressure of 140 Kpa was found to be the optimal pressure for fiber extrusion among the tested values.

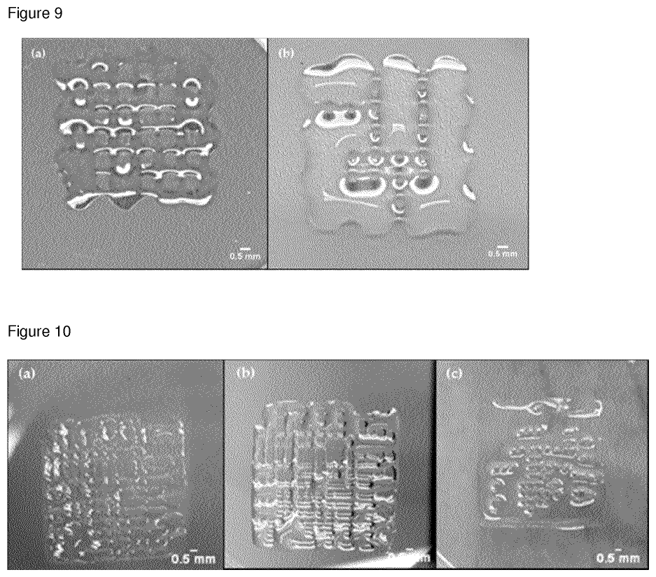

Printing was also tested with κ% ox-alg with 2% (w/v) and a lower concentration (1% (w/v)) of the 10% ox-alg. Since these formulation form softer and less viscous gels, smaller diameter needle and low extrusion pressures were tried for printing. Though both formulations showed promising 2-layered constructs, the integrity of the structure was compromised owing to their softer nature (Figures of printed structures are shown in FIGS. 9 & 10).

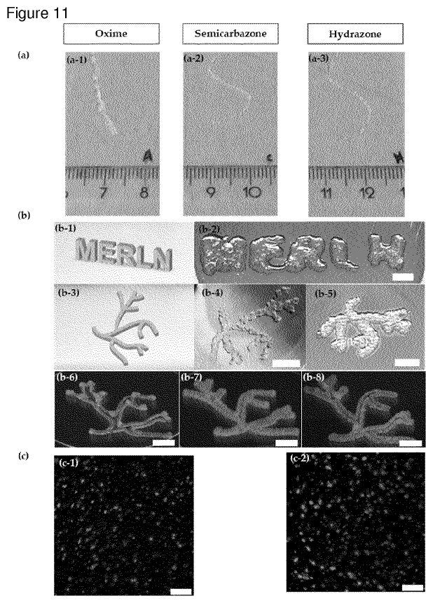

Our aims to create soft tissue constructs prompted the exploration of either more life-like or solid structures. Utilizing the optimized pressure (140 kPa) and deposition speed (5 mm/s), more complex and self-supporting structures were printed with 10% ox-alg gels with oxime, semicarbazone, and hydrazone different crosslinkers by extrusion through a 25G needle. FIG. 11b shows printed vascular tree and MERLN structures using 10% oxidized alginate with hydrazone crosslinks. From top left clockwise: 3D model of vascular tree, printed vascular tree (2 mm thickness), printed vascular tree (6 mm thickness), printed MERLN structure (6 mm thickness), and 3D model of MERLN structure, scale bar, 5 mm

While the recreation of the vascular tree was found possible, the printing of sharp angels and closely spaced or parallel lines resulted in occlusion and merging as found in the MERLN name. Although these bioinks could be optimized for better bioprint fidelity, the bioprinted structures are self-supporting while being deposited from dynamic and viscoelastic gels. Our initial optimization attempts with these materials formulations do show promising results, yet may need significant optimization, utilizing temperature sensitivity of hydrazone crosslinks or reinforcement from a Ca²⁺bath.

To increase bioprinting fidelity and print self-supporting structures with continuous deposition of material, printability was also tested by manually disrupting the gel network before printing. Preprinting network disruption allowed uniform deposition of material at seven mm/s using 150 kPa, and this modification resulted in a vascular structure with better printing resolution and uniformity (shown as b-6 in FIG. 11). This manual disruption method was also tested using 5% ox-alg (3 and 4% (w/v) and resulted in a similar result (shown as b-7 and b-8 in FIG. 11). However, these higher concentrations of hydrogel (3-4% (w/v)) needed significantly higher pressure (600 kPa) compared to 2% (w/v), 10% ox-alg (150 kPa). Further exploration of printing parameters and network dynamics modifications are expected to increase the printability of these materials.

The viability of 10% ox-alg (2% (w/v)) hydrazone gels with ATDC5 was evaluated 24 hrs after bioprinting and compared to the control, without bioprinting (shown in FIG. 11c). Very few dead cells were found, which is in agreement with the cell viability experiment carried out over 7 days (shown in FIG. 6). 24 hrs after bioprinting, cell viability decreased, which could be attributed to the shear stress suffered by cells during bioprinting.

1. A hydrogel comprising an oxidized alginate containing aldehyde groups, wherein the oxidized alginate has a molecular weight Mw of 80,000 daltons or more and the oxidized alginate is crosslinked with an imine type crosslinker selected from the group consisting of:
an alkoxy compound having formula (I): 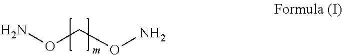
wherein m is 2 to 12,
a semicarbazide compound having formula (II): 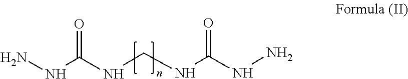
wherein n is 2 to 12, and
a hydrazide compound having formula (III): 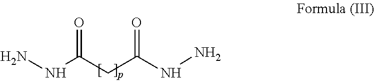
wherein p is 2 to 12;
wherein 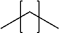
represents an alkylene group having m, n or p carbon atoms, wherein 1 or more carbon atoms can be replaced by a heteroatom selected from O, S and N;
a compound having formula (IV): 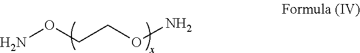
wherein x is 1 to 20;
a compound having formula (V): 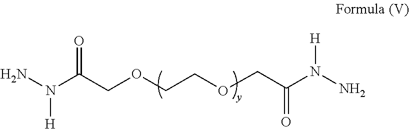
wherein y is 1 to 20;
a compound having formula (VI): 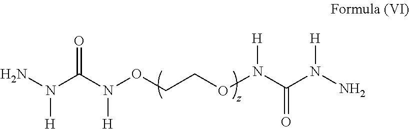
wherein z is 1 to 20;
a compound having formula (VII) 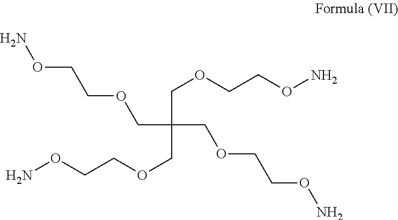 [SMILES: NOCCOCC(COCCON)(COCCON)COCCON]
a compound having formula (VIII) 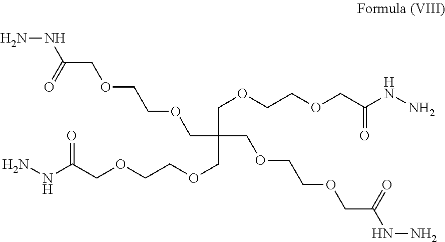 [SMILES: NNC(=O)COCCOCC(COCCOCC(=O)NN)(COCCOCC(=O)NN)COCCOCC(=O)NN]
a compound having formula (IX): 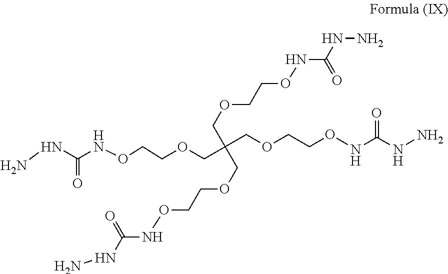 [SMILES: NNC(=O)NOCCOCC(COCCONC(=O)NN)(COCCONC(=O)NN)COCCONC(=O)NN]
a compound having formula (X): 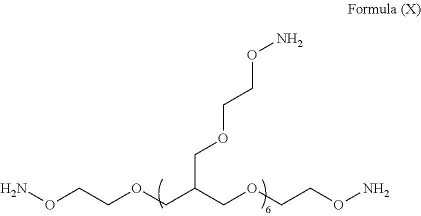
a compound having formula (XI) 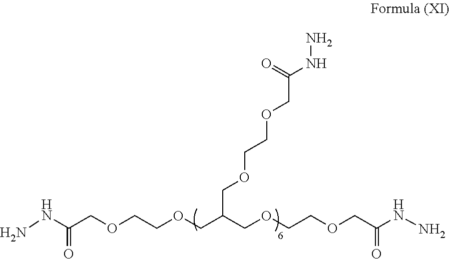
and
a compound having formula (XII):

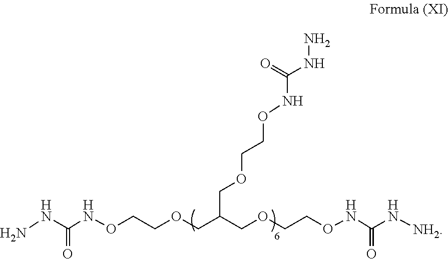

2. The hydrogel according to claim 1, wherein 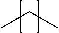
represents an alkylene group having m, n or p carbon atoms.

3. The hydrogel according to claim 1, wherein the oxidized alginate has a molecular weight M_{w} of 100,000 daltons or more.

4. The hydrogel according to claim 1, wherein the oxidized alginate is crosslinked with an imine type crosslinker selected from the group consisting of the compound having formula (I), the compound having formula (II) and the compound having formula (III).

5. The hydrogel according to claim 4, wherein the oxidized alginate is crosslinked with an imine type crosslinker having a structure:

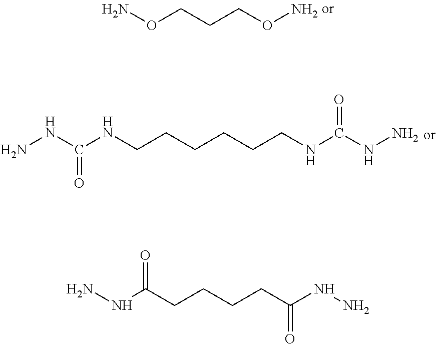 [SMILES: NOCCCON ; NNC(=O)NCCCCCCNC(=O)NN ; NNC(=O)CCCCC(=O)NN]

6. The hydrogel according to claim 1, wherein the oxidation level is 2% to 20%.

7. The hydrogel according to claim 1, wherein the alginate is 2% to 20% cross-linked.

8. The hydrogel according to claim 1, containing human or animal cells.

9. A method comprising:
using the hydrogel according to claim 1 to grow human or animal cells.

10. A reservoir containing the hydrogel according to claim 1, wherein the reservoir has a volume of at least 1 mL.

11. A method for manufacturing a 3D hydrogel structure comprising the steps of:
a) providing a suspension of an oxidized alginate containing aldehyde groups in a medium, wherein the oxidized alginate has a molecular weight M_{w} of 50,000 daltons or more;
b) adding an imine type crosslinker selected from the group consisting of an alkoxy compound having formula (I): 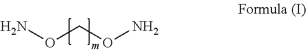
wherein m is 2 to 12,
a semicarbazide compound having formula (II): 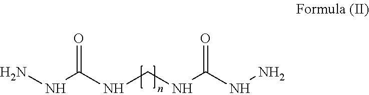
wherein n is 2 to 12, and
a hydrazide compound having formula (III): 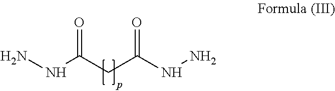
wherein p is 2 to 12
wherein 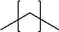
represents an alkylene group having m, n or p carbon atoms, wherein 1 or more carbon atoms can be replaced by a heteroatom selected from O, S and N;
a compound having formula (IV): 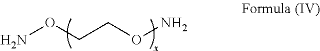
wherein x is 1 to 20;
a compound having formula (V): 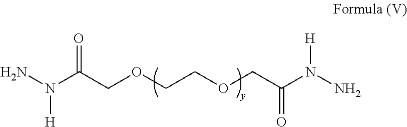
wherein y is 1 to 20;
a compound having formula (VI): 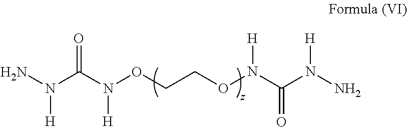
wherein z is 1 to 20;
a compound having formula (VII) 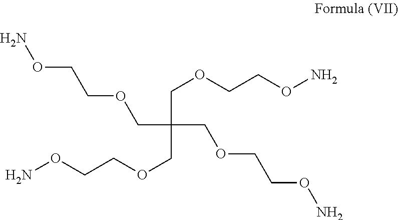 [SMILES: NOCCOCC(COCCON)(COCCON)COCCON]
a compound having formula (VIII) 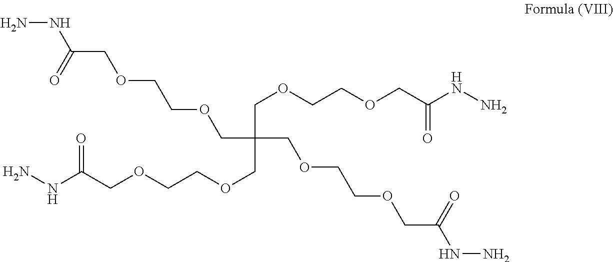 [SMILES: NNC(=O)COCCOCC(COCCOCC(=O)NN)(COCCOCC(=O)NN)COCCOCC(=O)NN]
a compound having formula (IX): 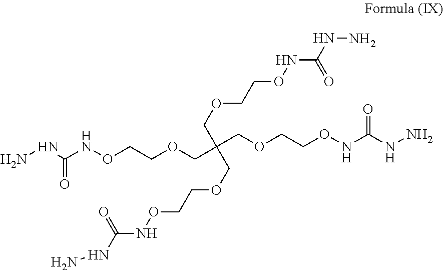 [SMILES: NNC(=O)NOCCOCC(COCCONC(=O)NN)(COCCONC(=O)NN)COCCONC(=O)NN]
a compound having formula (X): 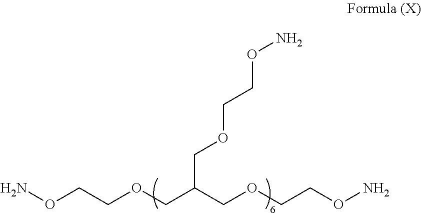
a compound having formula (XI) 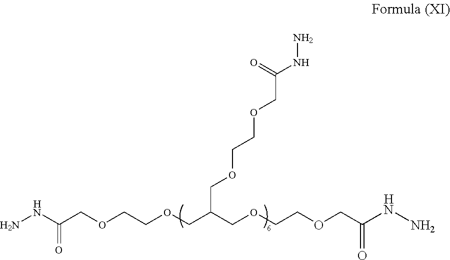
and
a compound having formula (XII): 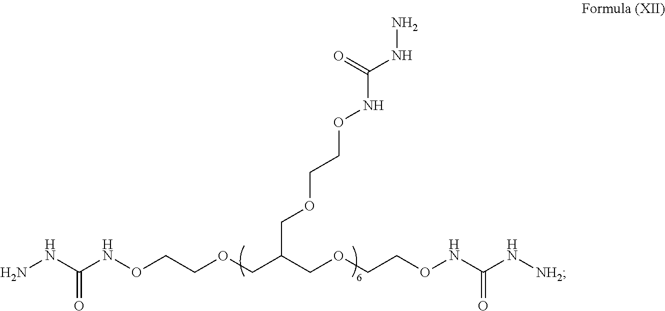
c) allowing a gel to form;
d) depositing the gel obtained in step c) layer by layer on a surface according to a predefined structure.

12. The method according to claim 11, wherein 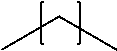
represents an alkylene group having m, n or p carbon atoms.

13. The method according to claim 11, wherein the imine type crosslinker is selected from a compound having a structure of Formula (I), Formula (II) or Formula (III).

14. The method according to claim 13, wherein the imine type crosslinker is selected from a compound having a structure:

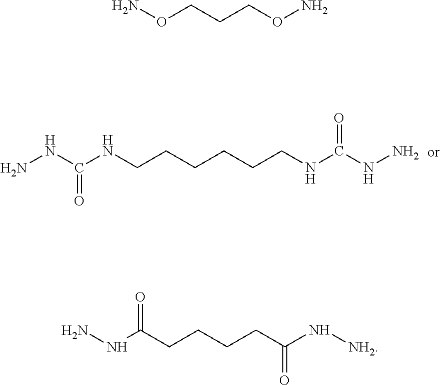 [SMILES: NOCCCON ; NNC(=O)NCCCCCCNC(=O)NN ; NNC(=O)CCCCC(=O)NN]

15. The method according to claim 11, wherein step a) further includes adding cells to the oxidized alginate containing aldehyde groups.
